(19) United States
(12) Patent Application Publication
(10) Pub. No.: US 2024/0165069 A1
(43) Pub. Date: May 23, 2024

(54) METHODS FOR TREATING DYSGEUSIA AND OTHER LINGERING SYMPTOMS ASSOCIATED WITH SEVERE ACUTE RESPIRATORY SYNDROME CORONAVIRUS 2 INFECTIONADN CORONAVIRUS DISEASE 2019

(71) Applicant: Nocendra, Inc., West Roxbury, MA (US)

(72) Inventors: Christopher R. Schuster, Groton, MA (US); Wesley D. Blakeslee, Westminster, MD (US)

(21) Appl. No.: 18/546,997

(22) PCT Filed: Feb. 18, 2022
(86) PCT No.: PCT/US22/16969

§ 371 Date: Aug. 18, 2023

Related U.S. Application Data
(60) Provisional application No. 63/151,202, filed on Feb. 19, 2021.

(51) Int. Cl. A61K 31/232; A61K 31/202

(52) U.S. Cl. CPC A61K 31/232; A61K 31/202

(57) ABSTRACT

Methods for treating one or more symptoms associated with a severe acute respiratory syndrome coronavirus 2 (SARS-CoV-2) infection and/or coronavirus disease 2019 (COVID-19), including lingering dysgeusia, by administering a lipoxin analog, such as BLXA₄, to a subject in need of treatment, including post-COVID-19 subjects.

### BACKGROUND

Coronavirus disease 2019 (COVID-19) is a highly contagious respiratory disease caused by the severe acute respiratory syndrome coronavirus 2 (SARS-CoV-2). COVID-19 is characterized by an initial robust cytokine storm as a response to infection with SARS-CoV-2, leading to an eicosanoid storm and subsequent organ damage. According to the World Health Organization (WHO), more than 50% of symptomatic COVID-19 patients present with lingering symptoms post-disease, which suggests low-level, but persistent chronic inflammation.

SARS CoV-2 binds to angiotensin-converting enzyme 2 (ACE2) as the critical host-cell receptor, which in turn leads to a loss of ACE2. Kuba et al., 2005; Wan et al., 2020; Li 2016. ACE2 confers protection against organ damage and has protective actions in hypertension, diabetes, cardiovascular disease and periodontitis. Gurkan et al., 2009a; Gurkan et al., 2009b; Cheng et al., 2020; Santos et al., 2015. Lack of ACE2 activity has been directly linked to the development of SARS. Kuba et al., 2005; Imai et al., 1966.

ACE2 is a key regulator of the renin-angiotensin system (RAS); it controls the abundance of angiotensin II (Ang II), the principal effector of the RAS, by generating Ang [1-7] that in turn signals through the protective Mas receptor. An imbalance of the pro- and anti-inflammatory arms of RAS has been associated with pathological states characterized by low-level chronic inflammation, Srivastava et al., 2019, with increased Ang II levels and reduced Ang [1-7] levels thought to exacerbate neuroinflammation. Valiuddin et al., 2020.

ACE2 receptors are expressed in the epithelium of taste buds and salivary glands in humans. Xu et al., 2020; Sakaguchi et al., 2020. Chemosensory dysfunction, including new early-onset dysgeusia (altered taste perception), is a common occurrence in SARS CoV-2 infection, Valiuddin et al., 2020, and is considered a good disease biomarker. Giacomelli et al., 2020. Dysgeusia is a common lingering symptom of COVID-19 after resolution of the disease, Lechien et al., 2020; Tong et al., 2020; Lozada-Nur et al., 2020. Further, approximately 50% of persistent dysgeusia cases have been associated with neuroinflammation (WHO, Valiuddin et al., 2020). Accordingly, there is a need for pharmacological strategies to address regulation of SARS CoV-2 mediated chronic inflammation, including dysgeusia.

### SUMMARY

In some aspects, the presently disclosed subject matter provides a method for treating one or more symptoms associated with a severe acute respiratory syndrome coronavirus 2 (SARS-CoV-2) infection and/or Coronavirus disease 2019 (COVID-19), the method comprising administering a therapeutically effective amount of a specialized proresolving mediator (SPM) to a subject in need of treatment thereof to treat the SARS-CoV-2 infection and/or COVID-19.

In certain aspects, the subject is post-COVID-19.

In particular aspects, the one or more symptoms associated with SARS-CoV-2 and/or COVID-19 comprises dysgeusia. In more particular aspects, the dysgeusia includes one or more of hypogeusia, ageusia, and aliageusia. In yet more particular aspects, the dysgeusia comprises new, early-onset dysgeusia. In other aspects, the dysgeusia comprises lingering dysgeusia post-COVID-19. In certain aspects, the subject experiences a change in taste perception. In more certain aspects, the taste perception of the subject is restored.

In other aspects, the one or more symptoms associated with severe acute respiratory syndrome coronavirus 2 (SARS-CoV-2) infection or COVID-19 comprises a pulmonary function. In particular aspects, the pulmonary function comprises dyspnea or fibrosis.

In some aspects, the specialized proresolving mediator (SPM) is administered orally. In certain aspects, the specialized proresolving mediator (SPM) is administered as an oral topical rinse. In more certain aspects, the subject is administered a dose of the specialized proresolving mediator (SPM) in the range of about 1 to about 20 μg/mL. In yet more certain aspects, the dose selected from the group consisting of about 1 μg/mL, 5 μg/mL, 10 μg/mL, and 20 μg/mL.

In some aspects, the specialized proresolving mediator (SPM) is selected from the group consisting of a lipoxin, a resolvin, a protectin, a maresin, and analogs thereof. In certain aspects, the specialized proresolving mediator comprises a lipoxin analog. In more certain aspects, the lipoxin analog comprises an analog of lipoxin A₄ (LXA₄), lipoxin B₄ (LXB₄), 15-epi-lipoxin A₄ (15-epi-LXA₄) or 15-epi-lipoxin B₄ (15-epi-LXB₄).

In particular aspects, the lipoxin analog comprises 9,12-benzo-lipoxin A₄ (BLXA₄). In other aspects, the lipoxin analog comprises the methyl ester of 9,12-benzo-lipoxin A₄ (BLXA₄-ME).

Certain aspects of the presently disclosed subject matter having been stated hereinabove, which are addressed in whole or in part by the presently disclosed subject matter, other aspects will become evident as the description proceeds when taken in connection with the accompanying Examples as best described herein below.

### DETAILED DESCRIPTION

The presently disclosed subject matter now will be described more fully hereinafter, in which some, but not all embodiments of the inventions are shown. The presently disclosed subject matter may be embodied in many different forms and should not be construed as limited to the embodiments set forth herein; rather, these embodiments are provided so that this disclosure will satisfy applicable legal requirements. Indeed. many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims.

The pathogenesis of coronavirus disease 2019 (COVID-19) involves an inflammatory response to Severe Acute Respiratory Syndrome Coronavirus 2 (SARS-Cov-2) infection, which results in various sequelae that complicate patients' recovery and long-term overall health, especially in susceptible populations. While current therapeutic clinical trials are targeting the acute inflammatory response and associated complications in infected individuals, the majority of patients (approximately 80%) who recover from the acute phase of disease still face clinical challenges with persistent and debilitating sequelae. In a single center study in Italy, Carfi et al., 2020, worsened quality of life was observed among 44.1% of patients after COVID-19 disease who were currently virus free. Therefore, continued follow up and therapeutic approaches are critical to ameliorate these long-lasting and debilitating sequelae from a public health perspective.

Acute inflammation seen in COVID-19 disease involves hyperactivation of T cells, macrophages, and natural killer cells, and the overproduction of pro-inflammatory cytokines (e.g., TNF-α, IL-6, IL-1, IL-8, and MCP-1). Iannaccone et al., 2020; Cirillo et al., 2021. The hyper-inflammatory profile can be persistent even after resolution of the acute phase of the disease and drive the persistent sequelae mediated by the chronic inflammatory response.

The physiological process of resolution of inflammation is actively regulated, rather than the passive termination of inflammation, as was once widely believed. Levy et al., 2001; Serhan 2004; Bannenberg et al., 2005; Serhan and Savill, 2005. The crucial identification of the cellular events and molecular signals that determine the end of inflammation and beginning of resolution has led to a new appreciation of pathogenesis in inflammatory diseases. Serhan et al., 2000; Lawrence et al., 2002; Heasman et al., 2003; Serhan et al., 2007.

Most current therapeutic approaches attempt to block activation of inflammation using anti-inflammatory drugs that are pathway inhibitors. Kantarci et al., 2006. A rapidly emerging body of evidence demonstrates that endogenous pro-resolving lipid mediators actively participate in regulating host responses and orchestrate resolution of inflammation. Serhan et al., 2002; Hong et al., 2003. Such endogenously produced lipids, referred to as specialized proresolving mediators (SPMs), actively participate in dampening host responses to infection, leading to active resolution of the inflammatory process. Serhan and Levy, 2018. SPMs include the lipoxins (LXs), resolvins, protectins, and maresins, all with the potential to resolve inflammation by actively signaling metabolic and cellular events that mediate the return to homeostasis after inflammation. Serhan and Levy, 2018. They are potent mediators acting in the picomolar to nanomolar dose range, which is a desirable characteristic for drug development. SPMs can attenuate neuroinflammation, as well as inflammatory and neuropathic pain. Svensson et al., 2007; Lima-Garcia et al., 2011; Xu et al., 2010; Li et al., 2013. Previous studies have reported that lipoxin A₄ (LXA₄) and the ACE2, Ang [1-7], and its receptor Mas [ACE2-Ang-(1-7)-Mas] axis play important protective roles in lung and liver injury. Hu et al., 2017; Chen et al., 2018.

Lipoxins, the product of lipoxygenase interactions, are trihydroxy derivatives of arachidonic acid that actively drive resolution of inflammation. While most COVID-19 disease treatment and post-disease management focus on “anti-viral” and “anti-inflammatory” strategies, stimulating inflammation resolution is a novel host-centric therapeutic avenue. Specialized proresolving lipid mediators (SPMs), including lipoxins and resolvins, may help to treat post-COVID-19 chronic illnesses, which in a significant proportion of such patients may be driven by persistent chronic inflammation. Importantly, BLXA₄ is currently in drug development phase with safety and preliminary efficacy results in a local inflammatory disease, gingivitis and could be rapidly translated for the amelioration and management of complications of post-COVID-19 disease via resolution of inflammation.

Lipoxin A₄ (LXA₄) is a member of the SPM family that drives resolution of inflammation through active, receptor mediated pathways. Failure of resolution pathways has been implicated in acute and chronic inflammatory conditions, including metabolic diseases, lung inflammation, and periodontitis. Serhan et al., 2008. SPMs and their analogs, such as BLXA₄, offer promise as host modulating agents with feed-forward agonist actions, rather than non-specific inhibition of inflammatory pathway enzymes or receptor antagonists. Serhan, 2017.

For example, in a Phase 1 trial comprising 50 subjects receiving drug for the treatment of oral inflammation, BLXA₄, administered as an oral topical mouth rinse, was indistinguishable from placebo mouth rinse in safety profile. While not powered for efficacy, trends in the efficacy data suggested reversal of inflammation (ClinicalTrials.gov NCT02342691). Since lipoxins do not inhibit inflammation, but actively resolve inflammation without impairing the natural inflammatory response, there is no known increase in susceptibility to infection as observed with inhibitors of inflammation, Serhan et al., 2008. As a result, clearance of infection is more efficient in the presence of lipoxins and resolvins. Herrera et al., 2015. Beyond its promising clinical efficacy, topical BLXA₄ also elicited a pro-resolving lipid mediator shift in the serum of the subjects.

Dysgeusia (impaired taste) manifests in several forms including hypogeusia, reduced or diminished sense of taste, or ageusia, complete loss of taste, as well as aliageusia, when a typically pleasant-tasting food or drink begins to taste unpleasant (e.g., metallic taste). Dysgeusia, in these forms, has been identified as both an early and lingering symptom in patients with COVID-19 along with other disease symptoms including fatigue, cough, headache, fever, chills, loss of smell, diarrhea, congestion, dyspnea, nausea, sore throat, chest pain, abdominal pain, confusion and vomiting (WHO). Impaired taste occurs in greater than 50% of symptomatic COVID-19 cases and lingers in greater than 50% of patients after resolution of acute disease. Carfi et al., 2020.

Neuroinflammation has been suggested as a possible mechanism for dysgeusia. Lahiri and Ardila, 2020. Gustatory functions are closely linked to olfactory functions. An impairment of the olfactory system, resulting from direct damage to nonneuronal cells in the olfactory epithelium resulting from replication and accumulation of SARS-CoV-2, is thought to be related to high expression of ACE2 receptors. Butowt and Bilinska, 2020. Damage to olfactory epithelium also can result in taste disturbance.

It also has been suggested that the virus directly affects the peripheral neuronal response of the gustatory tract by direct damage of ACE2-expressing cells of the taste buds and peripheral taste neurosensory chemoreceptors. Finsterer and Stollberger, 2020. More recently, an inflammatory response pathway has been suggested. The oral mucosa is lined with ACE2 receptors, which are used by SARS-CoV-2 to enter epithelial cells and ductal epithelium of the salivary glands. Xu et al., 2020; Liu et al., 2011.

SARS-CoV-2 binds to ACE2 receptors in the oral mucosa triggering an inflammatory response leading to cellular and possibly genetic changes that alter taste. Wang et al., 2009. Wang et al., 2009, demonstrated that cells in taste buds are capable of induction of inflammation through cytokine signaling that may impact taste. Tissue hypoxia also has been implicated in tissue injury that can lead to alterations in taste. The resulting anemia and poor oxygen transport have been shown to induce dysgeusia. Yukawa et al., 2001.

It also has been suggested that SARS-CoV-2 may cause zinc chelation through immune mechanisms and inflammatory processes may result in acute hypozincemia or localized changes in cellular zinc homeostasis in oral gustatory cells similar to acquired zinc deficiency. Wessels et al., 2020.

Accordingly, in some embodiments, the presently disclosed subject matter provides a method for treating one or more symptoms associated with a severe acute respiratory syndrome coronavirus 2 (SARS-CoV-2) infection and/or Coronavirus disease 2019 (COVID-19), the method comprising administering a therapeutically effective amount of a specialized proresolving mediator (SPM) to a subject in need of treatment thereof to treat the SARS-CoV-2 infection and/or COVID-19.

In certain embodiments, the subject is post-COVID-19.

In particular embodiments, the one or more symptoms associated with SARS-CoV-2 and/or COVID-19 comprises dysgeusia. In more particular embodiments, the dysgeusia includes one or more of hypogeusia, ageusia, and aliageusia. In yet more particular embodiments, the dysgeusia comprises new, early-onset dysgeusia. In other embodiments, the dysgeusia comprises lingering dysgeusia post-COVID-19. In certain embodiments, the subject experiences a change in taste perception. In more certain embodiments, the taste perception of the subject is restored.

In other embodiments, the one or more symptoms associated with severe acute respiratory syndrome coronavirus 2 (SARS-CoV-2) infection or COVID-19 comprises a pulmonary function. In particular embodiments, the pulmonary function comprises dyspnea or fibrosis.

In some embodiments, the specialized proresolving mediator (SPM) is administered orally. In certain embodiments, the specialized proresolving mediator (SPM) is administered as an oral topical rinse. In more certain embodiments, the subject is administered a dose of the specialized proresolving mediator (SPM) in the range of about 1 to about 20 μg/mL. In yet more certain embodiments, the dose selected from the group consisting of about 1 μg/mL, 5 μg/mL, 10 μg/mL, and 20 μg/mL.

In some embodiments, the specialized proresolving mediator (SPM) is selected from the group consisting of a lipoxin, a resolvin, a protectin, a maresin, and analogs thereof. In certain embodiments, the specialized proresolving mediator comprises a lipoxin analog.

Lipoxin analogs suitable for use with the presently disclosed methods are disclosed in U.S. Pat. No. 7,700,650 for Lipoxin Analogs and Method for the Treatment of Periodontal Disease, to Van Dyke et al., issued Apr. 20, 2010.

In some embodiments, the lipoxin analog is a structural analog of a biostable lipoxin compound, such as lipoxin A₄, lipoxin B₄, or other related lipid mediator. The term structural analog as used herein means any molecule having the basic structural components of a lipoxin compound as provided herein below. Such lipoxin analogs retain lipoxin activity, but do not undergo the typical metabolic deactivation of the parent lipoxin compounds. Thus, the in vivo half-life of the lipoxin analogs is significantly greater than the half-life of the parent compounds. In particular embodiments, the lipoxin analogs include, but are not limited to, structural analogs of two series of lipoxins: LXA series (LXA₄/15-epi-LXA₄) and LXB series (LXB₄/15-epi-LXB₄), including a structural analog of lipoxin A₄ (LXA₄), lipoxin B₄ (LXB₄), 15-epi-lipoxin A₄ (15-epi-LXA₄) or 15-epi-lipoxin B₄ (15-epi-LXB₄). The chemical formulae of the lipoxins include:
LxA₄: 5S,6R,15S-trihydroxy-7E,9E,11Z,13E-eicosatetraenoic acid;
LxB₄: 5S,14R,15S-trihydroxy-6E,8Z,10E,12E-eicosatetraenoic acid;
15-epi-LxA₄: 5S,6R,15R-trihydroxy-7E,9E,11Z,13E-eicosatetraenoic acid; and
15-epi-LxB₄: 5S,14R,15R-trihydroxy-6E,8Z,10E,12E-eicosatrienoic acid.

Lipoxin analogs contain four major components: (a) the carboxyl component, (b) the diol component, (c) the tetraene component, and (d) the alcohol component. Each of these components can possess a number of structural variations and still retain the key features necessary for lipoxin activity. Preferred compounds of the present invention generally belong either to the LXA series or the LXB series and can have structural modifications in one or more of the above components. The following diagram provides the general formulas for lipoxin analogs of the LXA and LXB series:

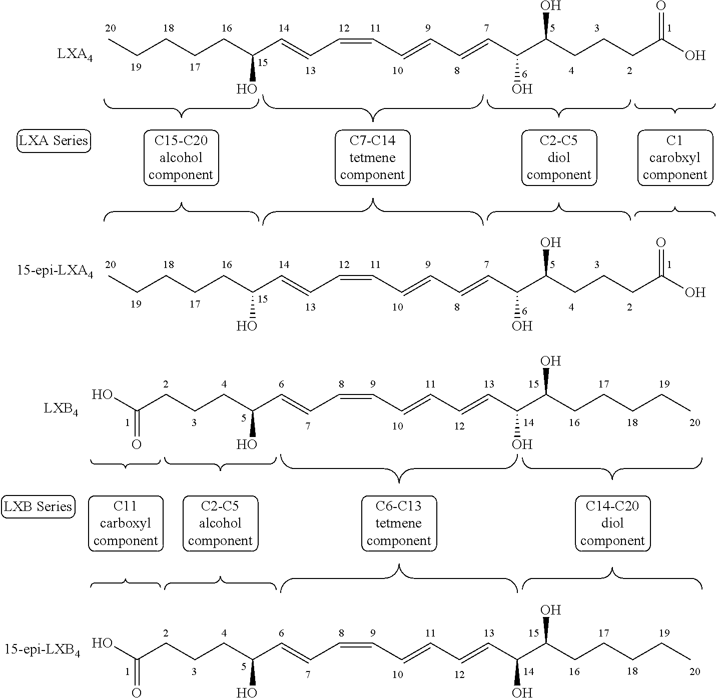

In some embodiments, the lipoxin analog has a general structural formula selected from the group consisting of:

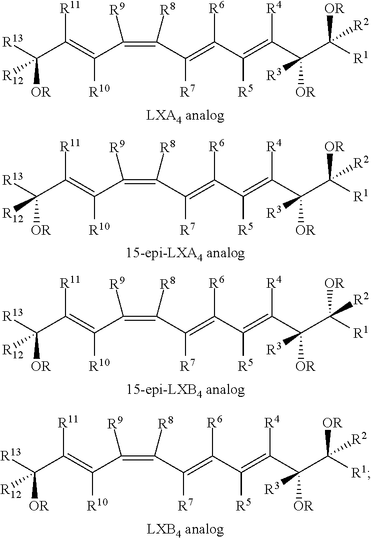

wherein:

R is hydrogen or a straight, branched, cyclic, saturated, or unsaturated alkyl;

R₁, R₂, R₁₂, R₁₃ are each independently selected from hydrogen, straight, branched, cyclic, saturated, or unsaturated alkyl having from 1 to 20 carbon atoms, substituted alkyl having from 1 to 20 carbon atoms, wherein the alkyl is substituted with one or more substituents selected from halo, hydroxy, lower alkoxy, aryloxy, amino, alkylamino, dialkylamino, acylamino, arylamino, hydroxyamino, alkoxyamino, alkylthio, arylthio, carboxy, carboxamido, carboalkoxy, aryl, and heteroaryl, substituted aryl or heteroaryl wherein the aryl or heteroaryl is substituted with one or more substituent selected from alkyl, cycloalkyl, alkoxy, halo, aryl, heteroaryl, carboxyl, and carboxamido, and a group Z-Y, wherein Z is a straight, branched, cyclic, saturated, or unsaturated alkyl having from 1 to 20 carbon atoms; substituted lower alkyl wherein the alkyl is substituted with one or more substituents selected from halo, hydroxy, lower alkoxy, aryloxy, amino, alkylamino, dialkylamino, acylamino, arylamino, hydroxyamino, alkoxyamino, alkylthio, arylthio, carboxy, carboxamido, carboalkoxy, aryl, and heteroaryl, substituted aryl or heteroaryl wherein the aryl or heteroaryl is substituted with one or more substituents selected from alkyl, cycloalkyl, alkoxy, halo, aryl, heteroaryl, carboxyl, and carboxamido, and Y is selected from hydrogen, alkyl, cycloalkyl, carboxyl, carboxamido, aryl, heteroaryl, substituted aryl or heteroaryl wherein the aryl or heteroaryl is substituted with one or more substituents selected from alkyl, cycloalkyl, alkoxy, halo, aryl, heteroaryl, carboxyl, and carboxamido;

R₃ is selected from hydrogen, straight, branched, cyclic, saturated, or unsaturated alkyl having from 1 to 20 carbon atoms, substituted alkyl having from 1 to 20 carbon atoms, wherein the alkyl is substituted with one or more substituents selected from the group consisting of halo, hydroxy, lower alkoxy, aryloxy, amino, alkylamino, dialkylamino, acylamino, arylamino, hydroxyamino, alkoxyamino, alkylthio, arylthio, carboxy, carboxamido, carboalkoxy, aryl, and heteroaryl; substituted aryl or heteroaryl, wherein the aryl or heteroaryl is substituted with one or more substituents selected from the group consisting of alkyl, cycloalkyl, alkoxy, halo, aryl, heteroaryl, carboxyl, and carboxamido; and

R₄, R₅, R₆, R₇, R₈, R₉, R₁₀, and R₁₁ are each independently selected from the group consisting of hydrogen; halo; straight, branched, cyclic, saturated, or unsaturated alkyl having from 1 to 20 carbon atoms; substituted alkyl having from 1 to 20 carbon atoms, wherein the alkyl is substituted with one or more substituents selected from halo, hydroxy, lower alkoxy, aryloxy, amino, alkylamino, dialkylamino, acylamino, arylamino, hydroxyamino, alkoxyamino, alkylthio, arylthio, carboxy, carboxamido, carboalkoxy, aryl, and heteroaryl; substituted aryl or heteroaryl wherein the aryl or heteroaryl are substituted with one or more substituent selected from alkyl, cycloalkyl, alkoxy, halo, aryl, heteroaryl, carboxyl, and carboxamido; or

R, R₁, R₂, R₃, R₄, R₅, R₆, R₇, R₈, R₉, R₁₀, R₁₁, R₁₂, and R₁₃ are connected to form one or more rings containing 3 to 20 carbon atoms, 1 to 6 oxygen atoms or 1 to 6 nitrogen atoms. A pair selected among the R₁, R₂, R₃, R₄, R₅, R₆, R₇, R₈, R₉, R₁₀, R₁₁, R₁₂, and R₁₃ groups can be replaced with a bond that generates a carbon-carbon double or triple bond or a ring.

In particular embodiments, the lipoxin analog is an LXA₄ series analog (left column) or a 15-epi-LXA₄ series analog (right column) selected from the group consisting of:

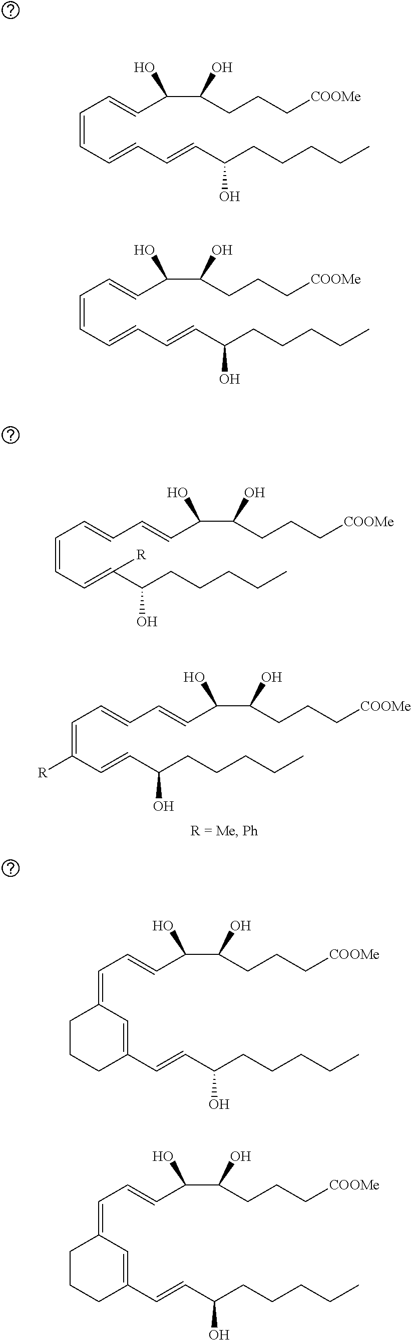

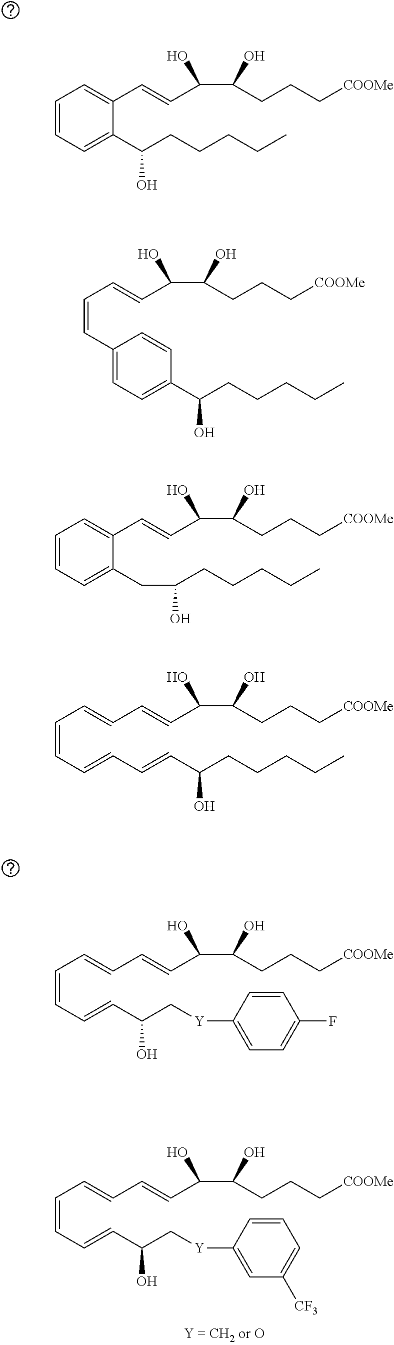

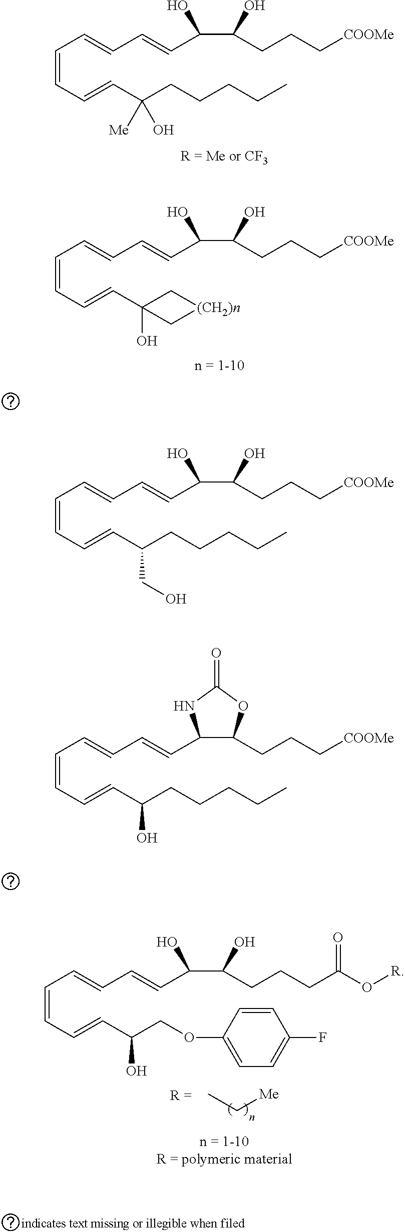

In other embodiments, the lipoxin analog is an LXB₄ series analog (left column) or a 15-epi-LXB₄ series analog (right column) selected from the group consisting of:

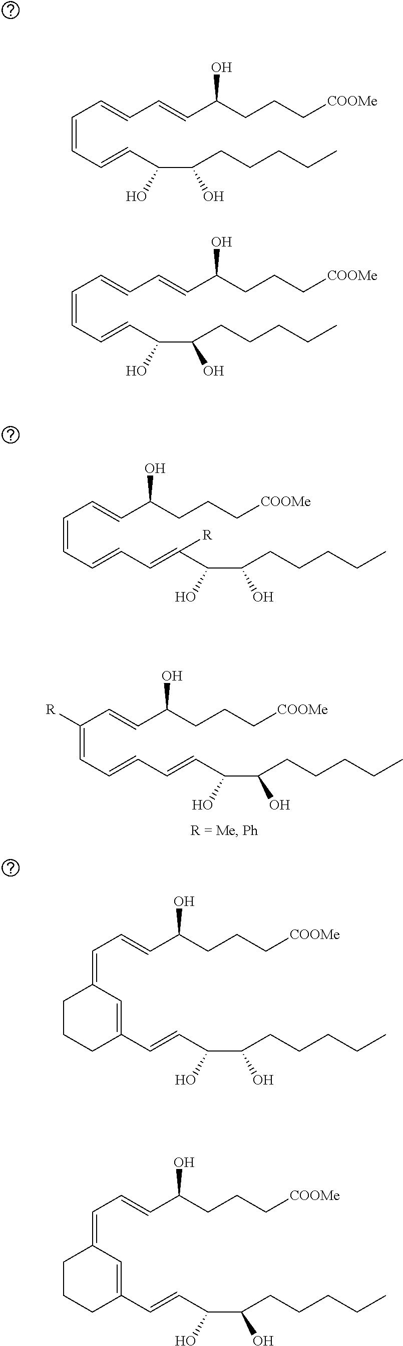

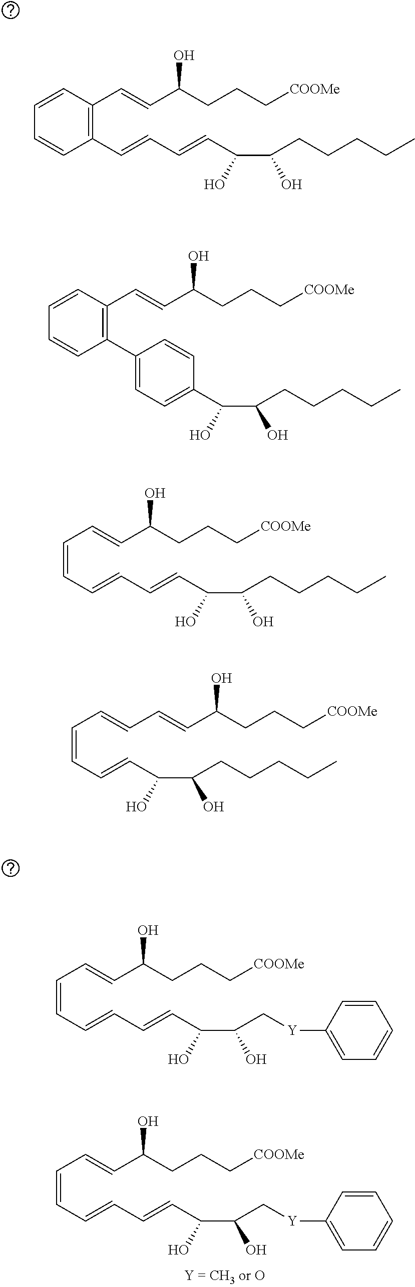

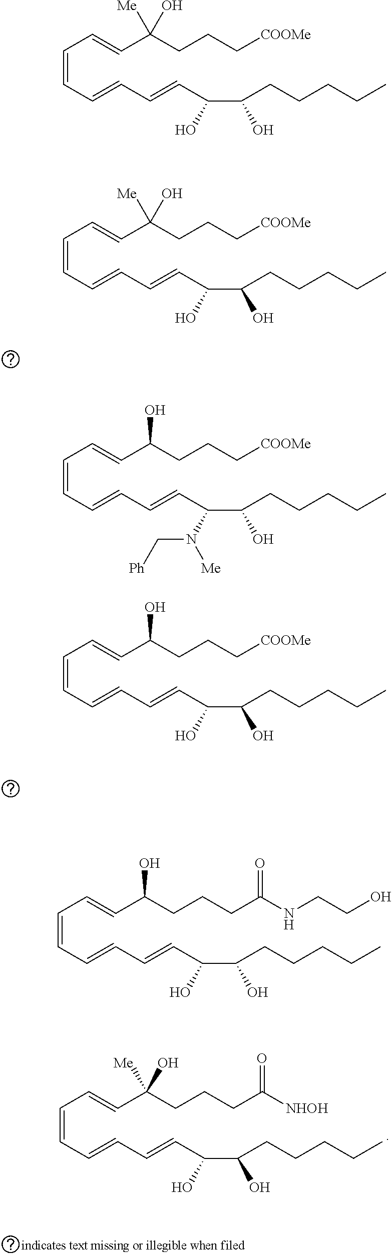

In particular embodiments, the lipoxin analog comprises 9,12-benzo-lipoxin A₄ (BLXA₄). In other embodiments, the lipoxin analog comprises the methyl ester of 9,12-benzo-lipoxin A₄ (BLXA₄-ME).

In certain embodiments, the lipoxin analog has the following structure:

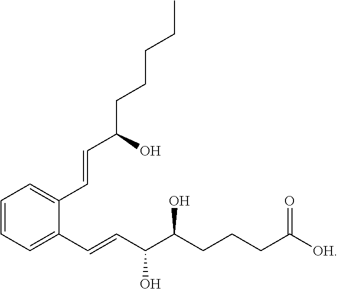 [SMILES: CCCCC[C@@H](O)/C=C/c1ccccc1/C=C/[C@@H](O)[C@@H](O)CCCC(=O)O]

In certain embodiments, the lipoxin analog has the following structure:

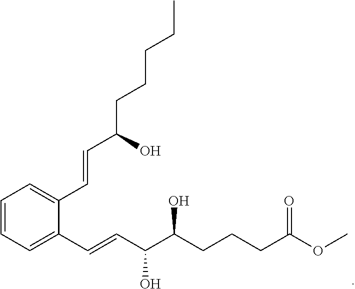 [SMILES: CCCCC[C@@H](O)/C=C/c1ccccc1/C=C/[C@@H](O)[C@@H](O)CCCC(=O)OC]

In certain embodiments, the lipoxin analog has the following structure:

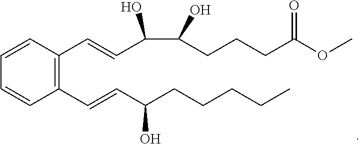 [SMILES: CCCCC[C@@H](O)/C=C/c1ccccc1/C=C/[C@@H](O)[C@@H](O)CCCC(=O)OC]

In some embodiments, the presently disclosed subject matter provides a composition comprising a lipoxin analog and one or more other components, wherein the composition is suitable for administration to a human or animal subject. The presently disclosed compounds or compositions thereof can be administered by any suitable means. In general, suitable means of administration include, but are not limited to, topical, transdermal, oral, sublingual, nasal, buccal, rectal, and parenteral (e.g., intravenous, subcutaneous or intramuscular) routes. In addition, the compositions can be incorporated into or covalently attached to polymers for topical use or for sustained delivery. The preferred method of administration is topical, for example, topical delivery to the oral cavity.

The presently disclosed compositions can be in any form including, but not limited to, solutions, suspensions, dispersions, ointments, creams, pastes, gels, powders, including tooth powders, toothpastes, lozenges, salve, chewing gum, mouth sprays, pastilles, sachets, mouthwashes, aerosols, tablets, capsules, transdermal patch, suppositories, and incorporated into floss. Preferred forms of the compositions are those that can be administered topically to the oral cavity. The presently disclosed compounds also can be incorporated into polymers, such as biodegradable polymers, micro- or nanoparticles, emulsions, liposomes, and the like, for the sustained release delivery.

The presently disclosed compositions can include other components including, but not limited to, pharmaceutical carriers, binders, fillers, flavorants, stabilizers, and one or more additional active ingredients.

As used herein, the term “treating” can include reversing, alleviating, inhibiting the progression of, preventing or reducing the likelihood of the disease, disorder, or condition to which such term applies, or one or more symptoms or manifestations of such disease, disorder or condition. Preventing refers to causing a disease, disorder, condition, or symptom or manifestation of such, or worsening of the severity of such, not to occur. Accordingly, the presently disclosed compounds can be administered prophylactically to prevent or reduce the incidence or recurrence of the disease, disorder, or condition.

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

While the following terms in relation to lipoxin analogs are believed to be well understood by one of ordinary skill in the art, the following definitions are set forth to facilitate explanation of the presently disclosed subject matter. These definitions are intended to supplement and illustrate, not preclude, the definitions that would be apparent to one of ordinary skill in the art upon review of the present disclosure.

The terms substituted, whether preceded by the term “optionally” or not, and substituent, as used herein, refer to the ability, as appreciated by one skilled in this art, to change one functional group for another functional group on a molecule, provided that the valency of all atoms is maintained. When more than one position in any given structure may be substituted with more than one substituent selected from a specified group, the substituent may be either the same or different at every position. The substituents also may be further substituted (e.g., an aryl group substituent may have another substituent off it, such as another aryl group, which is further substituted at one or more positions).

Where substituent groups or linking groups are specified by their conventional chemical formulae, written from left to right, they equally encompass the chemically identical substituents that would result from writing the structure from right to left, e.g., —CH₂O— is equivalent to —OCH₂—; —C(═O)O— is equivalent to —OC(═O)—; —OC(═O)NR— is equivalent to —NRC(═O)O—, and the like.

When the term “independently selected” is used, the substituents being referred to (e.g., R groups, such as groups R₁, R₂, and the like, or variables, such as “m” and “n”), can be identical or different. For example, both R₁ and R₂ can be substituted alkyls, or R₁ can be hydrogen and R₂ can be a substituted alkyl, and the like.

The terms “a,” “an,” or “a(n),” when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R-substituted, the moiety is substituted with at least one R substituent and each R substituent is optionally different.

A named “R” or group will generally have the structure that is recognized in the art as corresponding to a group having that name, unless specified otherwise herein. For the purposes of illustration, certain representative “R” groups as set forth above are defined below.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

Unless otherwise explicitly defined, a “substituent group,” as used herein, includes a functional group selected from one or more of the following moieties, which are defined herein:

The term hydrocarbon, as used herein, refers to any chemical group comprising hydrogen and carbon. The hydrocarbon may be substituted or unsubstituted. As would be known to one skilled in this art, all valencies must be satisfied in making any substitutions. The hydrocarbon may be unsaturated, saturated, branched, unbranched, cyclic, polycyclic, or heterocyclic. Illustrative hydrocarbons are further defined herein below and include, for example, methyl, ethyl, n-propyl, isopropyl, cyclopropyl, allyl, vinyl, n-butyl, tert-butyl, ethynyl, cyclohexyl, and the like.

The term “alkyl,” by itself or as part of another substituent, means, unless otherwise stated, a straight (i.e., unbranched) or branched chain, acyclic or cyclic hydrocarbon group, or combination thereof, which may be fully saturated, mono- or polyunsaturated and can include di- and multivalent groups, having the number of carbon atoms designated (i.e., C₁₋₁₀ means one to ten carbons, including 1, 2, 3, 4, 5, 6, 7, 8, 9, and 10 carbons). In particular embodiments, the term “alkyl” refers to C₁₋₂₀ inclusive, including 1, 2, 3, 4, 5, 6, 7, 8, 9, 10, 11, 12, 13, 14, 15, 16, 17, 18, 19, and 20 carbons, linear (i.e., “straight-chain”), branched, or cyclic, saturated or at least partially and in some cases fully unsaturated (i.e., alkenyl and alkynyl) hydrocarbon radicals derived from a hydrocarbon moiety containing between one and twenty carbon atoms by removal of a single hydrogen atom.

Representative saturated hydrocarbon groups include, but are not limited to, methyl, ethyl, n-propyl, isopropyl, n-butyl, isobutyl, sec-butyl, tert-butyl, n-pentyl, sec-pentyl, isopentyl, neopentyl, n-hexyl, sec-hexyl, n-heptyl, n-octyl, n-decyl, n-undecyl, dodecyl, cyclohexyl, (cyclohexyl)methyl, cyclopropylmethyl, and homologs and isomers thereof

“Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, acylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, cyano, and mercapto.

The term “heteroalkyl,” by itself or in combination with another term, means, unless otherwise stated, a stable straight or branched chain having from 1 to 20 carbon atoms or heteroatoms or a cyclic hydrocarbon group having from 3 to 10 carbon atoms or heteroatoms, or combinations thereof, consisting of at least one carbon atom and at least one heteroatom selected from the group consisting of O, N, P, Si and S, and wherein the nitrogen, phosphorus, and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, O—CH₃, —O—CH₂—CH₃, and —CN. Up to two or three heteroatoms may be consecutive, such as, for example, —CH₂—NH—OCH₃ and —CH₂—O—Si(CH₃)₃.

As described above, heteroalkyl groups, as used herein, include those groups that are attached to the remainder of the molecule through a heteroatom, such as —C(O)NR′, —NR′R″, —OR′, —SR, —S(O)R, and/or —S(O₂)R′. Where “heteroalkyl” is recited, followed by recitations of specific heteroalkyl groups, such as —NR′R or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, unsubstituted alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl, and fused ring systems, such as dihydro- and tetrahydronaphthalene, and the like.

The term “cycloalkylalkyl,” as used herein, refers to a cycloalkyl group as defined hereinabove, which is attached to the parent molecular moiety through an alkylene moiety, also as defined above, e.g., a C₁₋₂₀ alkylene moiety. Examples of cycloalkylalkyl groups include cyclopropylmethyl and cyclopentylethyl.

The terms “cycloheteroalkyl” or “heterocycloalkyl” refer to a non-aromatic ring system, unsaturated or partially unsaturated ring system, such as a 3- to 10-member substituted or unsubstituted cycloalkyl ring system, including one or more heteroatoms, which can be the same or different, and are selected from the group consisting of nitrogen (N), oxygen (O), sulfur (S), phosphorus (P), and silicon (Si), and optionally can include one or more double bonds.

The cycloheteroalkyl ring can be optionally fused to or otherwise attached to other cycloheteroalkyl rings and/or non-aromatic hydrocarbon rings. Heterocyclic rings include those having from one to three heteroatoms independently selected from oxygen, sulfur, and nitrogen, in which the nitrogen and sulfur heteroatoms may optionally be oxidized and the nitrogen heteroatom may optionally be quatemized. In certain embodiments, the term heterocylic refers to a non-aromatic 5-, 6-, or 7-membered ring or a polycyclic group wherein at least one ring atom is a heteroatom selected from O, S, and N (wherein the nitrogen and sulfur heteroatoms may be optionally oxidized), including, but not limited to, a bi- or tri-cyclic group, comprising fused six-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quatemized, and (iv) any of the above heterocyclic rings may be fused to an aryl or heteroaryl ring. Representative cycloheteroalkyl ring systems include, but are not limited to pyrrolidinyl, pyrrolinyl, imidazolidinyl, imidazolinyl, pyrazolidinyl, pyrazolinyl, piperidinyl, piperazinyl, indolinyl, quinuclidinyl, morpholinyl, thiomorpholinyl, thiadiazinanyl, tetrahydrofuranyl, and the like.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4- morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The terms “cycloalkylene” and “heterocycloalkylene” refer to the divalent derivatives of cycloalkyl and heterocycloalkyl, respectively.

An unsaturated hydrocarbon has one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups which are limited to hydrocarbon groups are termed “homoalkyl.”

More particularly, the term “alkenyl” as used herein refers to a monovalent group derived from a C₂₋₂₀ inclusive straight or branched hydrocarbon moiety having at least one carbon-carbon double bond by the removal of a single hydrogen molecule. Alkenyl groups include, for example, ethenyl (i.e., vinyl), propenyl, butenyl, 1-methyl-2-buten-1-yl, pentenyl, hexenyl, octenyl, allenyl, and butadienyl.

The term “cycloalkenyl” as used herein refers to a cyclic hydrocarbon containing at least one carbon-carbon double bond. Examples of cycloalkenyl groups include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “alkynyl” as used herein refers to a monovalent group derived from a straight or branched C₂₋₂₀ hydrocarbon of a designed number of carbon atoms containing at least one carbon-carbon triple bond. Examples of “alkynyl” include ethynyl, 2-propynyl (propargyl), 1-propynyl, pentynyl, hexynyl, and heptynyl groups, and the like.

The term “alkylene” by itself or a part of another substituent refers to a straight or branched bivalent aliphatic hydrocarbon group derived from an alkyl group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀); —CH═CH—CH═CH—; —CH═CH—CH₂—; —CH₂CH₂CH₂CH₂—, —CH₂CH═CHCH₂—, —CH₂CsCCH₂—, —CH₂CH₂CH(CH₂CH₂CH₃)CH₂—, —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons. Typically, an alkyl (or alkylene) group will have from 1 to 24 carbon atoms, with those groups having 10 or fewer carbon atoms being some embodiments of the present disclosure. A “lower alkyl” or “lower alkylene” is a shorter chain alkyl or alkylene group, generally having eight or fewer carbon atoms.

The term “heteroalkylene” by itself or as part of another substituent means a divalent group derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms also can occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′— represents both —C(O)OR′— and —R′OC(O)—.

The term “aryl” means, unless otherwise stated, an aromatic hydrocarbon substituent that can be a single ring or multiple rings (such as from 1 to 3 rings), which are fused together or linked covalently. The term “heteroaryl” refers to aryl groups (or rings) that contain from one to four heteroatoms (in each separate ring in the case of multiple rings) selected from N, O, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of above noted aryl and heteroaryl ring systems are selected from the group of acceptable substituents described below. The terms “arylene” and “heteroarylene” refer to the divalent forms of aryl and heteroaryl, respectively.

For brevity, the term “aryl” when used in combination with other terms (e.g., aryloxy, arylthioxy, arylalkyl) includes both aryl and heteroaryl rings as defined above. Thus, the terms “arylalkyl” and “heteroarylalkyl” are meant to include those groups in which an aryl or heteroaryl group is attached to an alkyl group (e.g., benzyl, phenethyl, pyridylmethyl, furylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like). However, the term “haloaryl,” as used herein is meant to cover only aryls substituted with one or more halogens.

Where a heteroalkyl, heterocycloalkyl, or heteroaryl includes a specific number of members (e.g. “3 to 7 membered”), the term “member” refers to a carbon or heteroatom.

Further, a structure represented generally by the formula:

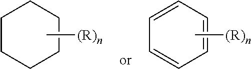

as used herein refers to a ring structure, for example, but not limited to a 3-carbon, a 4-carbon, a 5-carbon, a 6-carbon, a 7-carbon, and the like, aliphatic and/or aromatic cyclic compound, including a saturated ring structure, a partially saturated ring structure, and an unsaturated ring structure, comprising a substituent R group, wherein the R group can be present or absent, and when present, one or more R groups can each be substituted on one or more available carbon atoms of the ring structure. The presence or absence of the R group and number of R groups is determined by the value of the variable “n,” which is an integer generally having a value ranging from 0 to the number of carbon atoms on the ring available for substitution. Each R group, if more than one, is substituted on an available carbon of the ring structure rather than on another R group. For example, the structure above where n is 0 to 2 would comprise compound groups including, but not limited to:

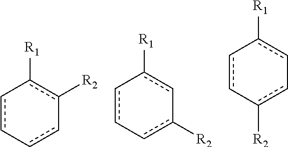

and the like.

A dashed line representing a bond in a cyclic ring structure indicates that the bond can be either present or absent in the ring. That is, a dashed line representing a bond in a cyclic ring structure indicates that the ring structure is selected from the group consisting of a saturated ring structure, a partially saturated ring structure, and an unsaturated ring structure.

The symbol ( [symbol] ) denotes the point of attachment of a moiety to the remainder of the molecule.

When a named atom of an aromatic ring or a heterocyclic aromatic ring is defined as being “absent,” the named atom is replaced by a direct bond.

Each of above terms (e.g. , “alkyl,” “heteroalkyl,” “cycloalkyl, and “heterocycloalkyl”, “aryl,” “heteroaryl,” “phosphonate,” and “sulfonate” as well as their divalent derivatives) are meant to include both substituted and unsubstituted forms of the indicated group. Optional substituents for each type of group are provided below.

Substituents for alkyl, heteroalkyl, cycloalkyl, heterocycloalkyl monovalent and divalent derivative groups (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″)═NR′″, —S(O)R′, —S(O)₂R′, —S(O)₂NR′R″, —NRSO₂R′, —CN, CF₃, fluorinated C₁₋₄ alkyl, and —NO₂ in a number ranging from zero to (2m′+1), where m′ is the total number of carbon atoms in such groups. R′, R″, R′″ and R″″ each may independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. As used herein, an “alkoxy” group is an alkyl attached to the remainder of the molecule through a divalent oxygen. When a compound of the disclosure includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 4-, 5-, 6-, or 7- membered ring. For example, —NR′R″ is meant to include, but not be limited to, 1- pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for alkyl groups above, exemplary substituents for aryl and heteroaryl groups (as well as their divalent derivatives) are varied and are selected from, for example: halogen, —OR′, —NR′R″, —SR′, —SiR′R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″R′″)═NR″″, —NR—C(NR′R″)═NR′″—S(O)R′, —S(O)₂R′, —S(O)₂NR′R″, —NRSO₂R′, —CN and —NO₂, —R′, —N₃, —CH(Ph)₂, fluoro(C₁₋₄)alkoxo, and fluoro(C₁₋₄)alkyl, in a number ranging from zero to the total number of open valences on aromatic ring system; and where R′, R″, R′″ and R″″ may be independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound of the disclosure includes more than one R group, for example, each of the R groups is independently selected as are each R′, R″, R′″ and R″″ groups when more than one of these groups is present.

Two of the substituents on adjacent atoms of aryl or heteroaryl ring may optionally form a ring of the formula —T—C(O)—(CRR′)_{q}—U—, wherein T and U are independently —NR—, —O—, —CRR′— or a single bond, and q is an integer of from 0 to 3. Alternatively, two of the substituents on adjacent atoms of aryl or heteroaryl ring may optionally be replaced with a substituent of the formula —A—(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4.

One of the single bonds of the new ring so formed may optionally be replaced with a double bond. Alternatively, two of the substituents on adjacent atoms of aryl or heteroaryl ring may optionally be replaced with a substituent of the formula —(CRR′)ₛ—X′—(C″R′″)_{d}—, where s and d are independently integers of from 0 to 3, and X′ is —O—, —NR′—, —S—, —S(O)—, —S(O)₂—, or —S(O)₂NR′—. The substituents R, R′, R″ and R′″ may be independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “acyl” refers to an organic acid group wherein the —OH of the carboxyl group has been replaced with another substituent and has the general formula RC(═O)—, wherein R is an alkyl, alkenyl, alkynyl, aryl, carbocylic, heterocyclic, or aromatic heterocyclic group as defined herein). As such, the term “acyl” specifically includes arylacyl groups, such as a 2-(furan-2-yl)acetyl)- and a 2-phenylacetyl group. Specific examples of acyl groups include acetyl and benzoyl. Acyl groups also are intended to include amides, —RC(═O)NR′, esters, —RC(═O)OR′, ketones, —RC(═O)R′, and aldehydes, —RC(═O)H.

The terms “alkoxyl” or “alkoxy” are used interchangeably herein and refer to a saturated (i.e., alkyl—O—) or unsaturated (i.e., alkenyl-O— and alkynyl-O—) group attached to the parent molecular moiety through an oxygen atom, wherein the terms “alkyl,” “alkenyl,” and “alkynyl” are as previously described and can include C₁₋₂₀ inclusive, linear, branched, or cyclic, saturated or unsaturated oxo-hydrocarbon chains, including, for example, methoxyl, ethoxyl, propoxyl, isopropoxyl, n-butoxyl, sec-butoxyl, tert-butoxyl, and n-pentoxyl, neopentoxyl, n-hexoxyl, and the like.

The term “alkoxyalkyl” as used herein refers to an alkyl-O-alkyl ether, for example, a methoxyethyl or an ethoxymethyl group.

“Aryloxyl” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

“Aralkyl” refers to an aryl-alkyl-group wherein aryl and alkyl are as previously described, and included substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl, i.e., C₆H₅—CH₂—O—. An aralkyloxyl group can optionally be substituted.

“Alkoxycarbonyl” refers to an alkyl-O—C(═O)— group. Exemplary alkoxycarbonyl groups include methoxycarbonyl, ethoxycarbonyl, butyloxycarbonyl, and tert-butyloxycarbonyl.

“Aryloxycarbonyl” refers to an aryl-O—C(═O)— group. Exemplary aryloxycarbonyl groups include phenoxy- and naphthoxy-carbonyl.

“Aralkoxycarbonyl” refers to an aralkyl-O—C(═O)— group. An exemplary aralkoxycarbonyl group is benzyloxycarbonyl.

“Carbamoyl” refers to an amide group of the formula —C(═O)NH₂. “Alkylcarbamoyl” refers to a R′RN—C(═O)— group wherein one of R and R′ is hydrogen and the other of R and R′ is alkyl and/or substituted alkyl as previously described. “Dialkylcarbamoyl” refers to a R′RN—C(═O)— group wherein each of R and R′ is independently alkyl and/or substituted alkyl as previously described.

The term carbonyldioxyl, as used herein, refers to a carbonate group of the formula —O—C(═O)—OR.

“Acyloxyl” refers to an acyl-O— group wherein acyl is as previously described.

The term “amino” refers to the —NH₂ group and also refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups, respectively.

An “aminoalkyl” as used herein refers to an amino group covalently bound to an alkylene linker. More particularly, the terms alkylamino, dialkylamino, and trialkylamino as used herein refer to one, two, or three, respectively, alkyl groups, as previously defined, attached to the parent molecular moiety through a nitrogen atom. The term alkylamino refers to a group having the structure —NHR' wherein R′ is an alkyl group, as previously defined; whereas the term dialkylamino refers to a group having the structure —NR′R″, wherein R′ and R″ are each independently selected from the group consisting of alkyl groups. The term trialkylamino refers to a group having the structure —NR′R″R″', wherein R′, R″, and R″ are each independently selected from the group consisting of alkyl groups. Additionally, R′, R″, and/or R″ taken together may optionally be —(CH₂)k— where k is an integer from 2 to 6. Examples include, but are not limited to, methylamino, dimethylamino, ethylamino, diethylamino, diethylaminocarbonyl, methylethylamino, isopropylamino, piperidino, trimethylamino, and propylamino.

The amino group is —NR′R″, wherein R′ and R″ are typically selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

The terms alkylthioether and thioalkoxyl refer to a saturated (i.e., alkyl-S—) or unsaturated (i.e., alkenyl-S— and alkynyl-S—) group attached to the parent molecular moiety through a sulfur atom. Examples of thioalkoxyl moieties include, but are not limited to, methylthio, ethylthio, propylthio, isopropylthio, n-butylthio, and the like.

“Acylamino” refers to an acyl-NH— group wherein acyl is as previously described. “Aroylamino” refers to an aroyl-NH— group wherein aroyl is as previously described.

The term “carbonyl” refers to the —C(═O)— group, and can include an aldehyde group represented by the general formula R—C(═O)H.

The term “carboxyl” refers to the —COOH group. Such groups also are referred to herein as a “carboxylic acid” moiety.

The term “cyano” refers to the —C≡N group.

The terms “halo,” “halide,” or “halogen” as used herein refer to fluoro, chloro, bromo, and iodo groups. Additionally, terms such as “haloalkyl,” are meant to include monohaloalkyl and polyhaloalkyl. For example, the term “halo(C₁₋₄)alkyl” is mean to include, but not be limited to, trifluoromethyl, 2,2,2-trifluoroethyl, 4-chlorobutyl, 3-bromopropyl, and the like.

The term “hydroxyl” refers to the —OH group.

The term “hydroxyalkyl” refers to an alkyl group substituted with an —OH group.

The term “mercapto” refers to the —SH group.

The term “oxo” as used herein means an oxygen atom that is double bonded to a carbon atom or to another element.

The term “nitro” refers to the —NO₂ group.

The term “thio” refers to a compound described previously herein wherein a carbon or oxygen atom is replaced by a sulfur atom.

The term “sulfate” refers to the —SO₄ group.

The term thiohydroxyl or thiol, as used herein, refers to a group of the formula —SH.

More particularly, the term “sulfide” refers to compound having a group of the formula —SR.

The term “sulfone” refers to compound having a sulfonyl group —S(O₂)R.

The term “sulfoxide” refers to a compound having a sulfinyl group —S(O)R

The term ureido refers to a urea group of the formula —NH—CO—NH₂.

Throughout the specification and claims, a given chemical formula or name shall encompass all tautomers, congeners, and optical- and stereoisomers, as well as racemic mixtures where such isomers and mixtures exist.

Certain compounds of the present disclosure may possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)-or (S)- or, as D- or L- for amino acids, and individual isomers are encompassed within the scope of the present disclosure. The compounds of the present disclosure do not include those which are known in art to be too unstable to synthesize and/or isolate. The present disclosure is meant to include compounds in racemic, scalemic, and optically pure forms. Optically active (R)- and (S)- , or D- and L-isomers may be prepared using chiral synthons or chiral reagents, or resolved using conventional techniques. When the compounds described herein contain olefenic bonds or other centers of geometric asymmetry, and unless specified otherwise, it is intended that the compounds include both E and Z geometric isomers.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

It will be apparent to one skilled in the art that certain compounds of this disclosure may exist in tautomeric forms, all such tautomeric forms of the compounds being within the scope of the disclosure. The term “tautomer,” as used herein, refers to one of two or more structural isomers which exist in equilibrium and which are readily converted from one isomeric form to another.

Unless otherwise stated, structures depicted herein are also meant to include compounds which differ only in the presence of one or more isotopically enriched atoms. For example, compounds having the present structures with the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of atoms that constitute such compounds. For example, the compounds may be radiolabeled with radioactive isotopes, such as for example tritium (3 H), iodine-125 (¹²⁵I) or carbon-14 (¹⁴C). All isotopic variations of the compounds of the present disclosure, whether radioactive or not, are encompassed within the scope of the present disclosure.

The compounds of the present disclosure may exist as salts. The present disclosure includes such salts. Examples of applicable salt forms include hydrochlorides, hydrobromides, sulfates, methanesulfonates, nitrates, maleates, acetates, citrates, fumarates, tartrates (e.g. (+)-tartrates, (−)-tartrates or mixtures thereof including racemic mixtures, succinates, benzoates and salts with amino acids such as glutamic acid. These salts may be prepared by methods known to those skilled in art. Also included are base addition salts such as sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent or by ion exchange. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present disclosure contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

In addition to salt forms, the present disclosure provides compounds, which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the makeup of the pharmaceutical composition, the target tissue, and the like.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount, or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### BLXA₄ for Treating Lingering Symptoms of COVID-19

Without wishing to be bound to any one particular theory, it is thought that unresolved chronic inflammation is responsible for persistent taste alterations and that BLXA₄, a pro-resolution lipid mediator, can restore taste perception. Accordingly, the efficacy of a BLXA₄ oral rinse for treating lingering symptoms of COVID-19, e.g., dysgeusia, can be determined. For example, a subject afflicted with COVID-19, either pre-, post-, or during infection, can be administered a therapeutically effective amount of a BLXA₄ oral rinse. In some embodiments, the administration is daily. In some embodiments, the efficacy of a BLXA₄ oral rinse for treating lingering symptoms of COVID-19 can be determined over a period of time, e.g., over a one-month trial.

Exploratory outcomes can include clinical measures of oral inflammation and other persistent disease symptoms, such as residual lung dysfunction and radiographic findings. Biological samples can be collected and analyzed for evidence and severity of oral inflammation to determine potential pathways of action.

In some embodiments, the subject is post-COVID-19, but who is no longer considered to be infectious. Post-COVID-19 subjects afflicted with lingering dysgeusia can be administered an oral rinse comprising between about 1 μg/mL to 20 μg/mL BLXA₄, including about 1, 2, 3, 4, 5, 6, 7, 8, 9, 10, 11, 12, 13, 14, 15, 16, 17, 18, 19, and 20 μg/mL BLXA₄. In particular embodiments, a post-COVID-19 subject afflicted with lingering dysgeusia can be administered an oral rinse comprising 1 μg/mL, 5 μg/mL, 10 μg/mL, or 20 μg/mL BLXA₄. An aliquot, e.g., fifteen milliliters, of rinse can be administered daily for a brief period of time, e.g., 45 seconds, over a period of time, e.g., for 28 days. Taste can be assessed periodically, e.g., weekly, using both standard objective measures, Coldwell et al., 2013, and self-reported changes. Giacomelli et a 1., 2020. Other physical evaluations, including pulmonary function (spirometry) will be recorded weekly. Oral assessments, sampling for inflammatory markers and microbiological profiles, and chest X-rays will be performed at baseline and 28 days.

### Example 2

### Impact of Daily BLXA₄ Treatment on SPM Profiles and Markers of the Renin-Angiotensin System (RAS)

Without wishing to be bound to any one particular theory, it is thought that daily BLXA₄ treatment will recover SPM and RAS profiles that facilitate resolution. To do so, inflammation in subject afflicted with lingering dysgeusia can be assessed by determining SPM and RAS profiles that favor pro-inflammation. Using validated, targeted mass-spectrometry-based assays, the components of the lipidomic profile can be quantified in serum and saliva and both the pro-inflammatory and protective arm of the renin-angiotensin system (RAS) can be quantified in plasma and saliva samples.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.
Lechien J. R., Chiesa-Estomba C. M., De Siati D. R., Horoi M, Le Bon S. D., Rodriguez A, Dequanter D, Blecic S, El Afia F, Distinguin L, Chekkoury-Idrissi Y, Hans S, Delgado I. L., Calvo-Henriquez C, Lavigne P, Falanga C, Barillari M. R., Cammaroto G, Khalife M, Leich P, Souchay C, Rossi C, Journe F, Hsieh J, Edjlali M, Carlier R, Ris L, Lovato A, De Filippis C, Coppee F, Fakhry N, Ayad T, Saussez S. Olfactory and gustatory dysfunctions as a clinical presentation of mild-to-moderate forms of the coronavirus disease (COVID-19): a multicenter European study. European archives of oto-rhino-laryngology : official journal of the European Federation of Oto-Rhino-Laryngological Societies. 2020; 277(8):2251-61.
Tong J. Y., Wong A, Zhu D, Fastenberg J. H., Tham T. The Prevalence of Olfactory and Gustatory Dysfunction in COVID-19 Patients: A Systematic Review and Meta-analysis. Otolaryngol Head Neck Surg. 2020; 163(1):3-11.
Lozada-Nur F, Chainani-Wu N, Fortuna G, Sroussi H. Dysgeusia in COVID-19: Possible Mechanisms and Implications. Oral Surg Oral Med Oral Pathol Oral Radiol. 2020; 130(3):344-6.
Valiuddin H. M., Kalajdzic A, Rosati J, Boehm K, Hill D. Update on Neurological Manifestations of SARS-CoV-2. West J Emerg Med. 2020.
Giacomelli A, Pezzati L, Conti F, Bernacchia D, Siano M, Oreni L, Rusconi S, Gervasoni C, Ridolfo AL, Rizzardini G, Antinori S, Galli M. Self-reported Olfactory and Taste Disorders in Patients With Severe Acute Respiratory Coronavirus 2 Infection: A Cross-sectional Study. Clinical infectious diseases : an official publication of the Infectious Diseases Society of America. 2020; 71(15):889-90.
Kuba K, Imai Y, Rao S, Gao H, Guo F, Guan B, Huan Y, Yang P, Zhang Y, Deng W, Bao L, Zhang B, Liu G, Wang Z, Chappell M, Liu Y, Zheng D, Leibbrandt A, Wada T, Slutsky A. S., Liu D, Qin C, Jiang C, Penninger J. M.. A crucial role of angiotensin converting enzyme 2 (ACE2) in SARS coronavirus-induced lung injury. Nature medicine. 2005; 11(8):875-9.
Wan Y, Shang J, Graham R, Baric R. S., Li F. Receptor Recognition by the Novel Coronavirus from Wuhan: an Analysis Based on Decade-Long Structural Studies of SARS Coronavirus. Journal of virology. 2020; 94(7).
Li F. Structure, Function, and Evolution of Coronavirus Spike Proteins. Annu Rev Virol. 2016; 3(1):237-61.
Xu H, Zhong L, Deng J, Peng J, Dan H, Zeng X, Li T, Chen Q. High expression of ACE2 receptor of 2019-nCoV on the epithelial cells of oral mucosa. Int J Oral Sci. 2020; 12(1): 8.
Sakaguchi W, Kubota N, Shimizu T, Saruta J, Fuchida S, Kawata A, Yamamoto Y, Sugimoto M, Yakeishi M, Tsukinoki K. Existence of SARS-CoV-2 Entry Molecules in the Oral Cavity. International journal of molecular sciences. 2020; 21(17).
Imai H, Lee K. T., Pastori S, Panlilio E, Florentin R, Thomas W. A.. Atherosclerosis in rabbits. Architectural and subcellular alterations of smooth muscle cells of aortas in response to hyperlipemia. Exp Mol Pathol. 1966; 5(3):273-310.
Gurkan A, Emingil G, Saygan B. H., Atilla G, Kose T, Baylas H, Berdeli A. Renin-angiotensin gene polymorphisms in relation to severe chronic periodontitis. Journal of clinical periodontology. 2009a; 36(3):204-11.
Gurkan A, Emingil G, Saygan B. H., Atilla G, Kose T, Baylas H, Berdeli A. Angiotensin-converting enzyme (ACE), angiotensinogen (AGT), and angiotensin II type 1 receptor (AT1R) gene polymorphisms in generalized aggressive periodontitis. Archives of oral biology. 2009b; 54(4):337-44.
Cheng H, Wang Y, Wang G. Q.. Organ-protective effect of angiotensin-converting enzyme 2 and its effect on the prognosis of COVID-19. Journal of medical virology. 2020; 92(7):726-30.
Santos C. F., Morandini A. C., Dionisio T. J., Faria F. A., Lima M. C., Figueiredo C. M., Colombini-Ishikiriama B. L., Sipert C. R., Maciel R. P., Akashi A. P., Souza G. P., Garlet G. P., Rodini C. O., Amaral S. L., Becari C, Salgado M. C., Oliveira E. B., Matus I, Didier D. N., Greene A. S. Functional Local Renin-Angiotensin System in Human and Rat Periodontal Tissue. PloS one. 2015; 10(8): e0134601.
Srivastava P, Badhwar S, Chandran DS, Jaryal AK, Jyotsna VP, Deepak K. K. Imbalance between Angiotensin II—Angiotensin (1-7) system is associated with vascular endothelial dysfunction and inflammation in type 2 diabetes with newly diagnosed hypertension. Diabetes Metab Syndr. 2019; 13(3):2061-8.
Serhan C. N., Levy B. D. Resolvins in inflammation: emergence of the pro-resolving superfamily of mediators. The Journal of clinical investigation. 2018; 128(7):2657-69.
Svensson C. I., Zattoni M, Serhan C. N. Lipoxins and aspirin-triggered lipoxin inhibit inflammatory pain processing. The Journal of experimental medicine. 2007; 204(2):245-52.
Lima-Garcia J. F., Dutra R. C., da Silva K, Motta E. M., Campos M. M., Calixto J. B. The precursor of resolvin D series and aspirin-triggered resolvin D1 display anti-hyperalgesic properties in adjuvant-induced arthritis in rats. British journal of pharmacology. 2011; 164(2):278-93.
Xu Z. Z., Zhang L, Liu T, Park J. Y., Berta T, Yang R, Serhan CN, Ji R. R. Resolvins RvE1 and RvD1 attenuate inflammatory pain via central and peripheral actions. Nature medicine. 2010; 16(5):592-7.
Li Q, Tian Y, Wang Z. F., Liu S. B., Mi W. L., Ma H. J., Wu G. C., Wang J, Yu J, Wang Y. Q. Involvement of the spinal NALP1 inflammasome in neuropathic pain and aspirin-triggered-15-epi-lipoxin A4 induced analgesia. Neuroscience. 2013; 254:230-40.
Hu Q, Hu Z, Chen Q, Huang Y, Mao Z, Xu F, Zhou X. BML-111 equilibrated ACE-AngII-AT1R and ACE2-Ang-(1-7)-Mas axis to protect hepatic fibrosis in rats. Prostaglandins Other Lipid Mediat. 2017; 131:75-82.
Chen Q. F., Kuang X. D., Yuan Q. F., Hao H, Zhang T, Huang Y. H., Zhou X. Y. Lipoxin A4 attenuates LPS-induced acute lung injury via activation of the ACE2-Ang-(1-7)-Mas axis. Innate immunity. 2018; 24(5):285-96.
Coldwell S. E., Mennella J. A., Duffy V. B., Pelchat M. L., Griffith J. W., Smutzer G, Cowart B. J., Breslin P. A., Bartoshuk L. M., Hastings L, Victorson D, Hoffman H. J. Gustation assessment using the NIH Toolbox. Neurology. 2013; 80(11 Suppl 3):S20-4.
Carfi A, Bernabei R, Landi F, Gemelli Against C-P-ACSG. Persistent Symptoms in Patients After Acute COVID-19. JAMA: the journal of the American Medical Association. 2020; 324(6): 603-5.
Iannaccone G, Scacciavillani R, Del Buono M. G., Camilli M, Ronco C, Lavie C. J., Abbate A, Crea F, Massetti M, Aspromonte N. Weathering the Cytokine Storm in COVID-19: Therapeutic Implications. Cardiorenal Med. 2020; 10(5):277-87.
Cirillo N, Bizzoca M. E., Lo Muzio E, Cazzolla A. P., Lo Muzio L. Gustatory dysfunction in COVID-19 patients: a rapid systematic review on 27,687 cases. Acta Odontol Scand. 2021:1-8.
Serhan C. N., Chiang N, Van Dyke T. E. Resolving inflammation: dual anti-inflammatory and pro-resolution lipid mediators. Nature reviews Immunology. 2008; 8(5):349-61.
Serhan C. N. Treating inflammation and infection in the 21st century: new hints from decoding resolution mediators and mechanisms. FASEB journal: official publication of the Federation of American Societies for Experimental Biology. 2017; 31(4):1273-88.
Herrera B. S., Hasturk H, Kantarci A, Freire M. O., Nguyen O, Kansal S, Van Dyke T. E. Impact of resolvin E1 on murine neutrophil phagocytosis in type 2 diabetes. Infection and immunity. 2015; 83(2): 792-801.
Lahiri D, Ardila A. COVID-19 Pandemic: A Neurological Perspective. Cureus. 2020; 12(4):e7889.
Butowt R, Bilinska K. SARS-CoV-2: Olfaction, Brain Infection, and the Urgent Need for Clinical Samples Allowing Earlier Virus Detection. ACS chemical neuroscience. 2020; 11(9):1200-3.
Finsterer J, Stollberger C. Causes of hypogeusia/hyposmia in SARS-CoV2 infected patients. Journal of medical virology. 2020; 92(10):1793-1794.
Liu L, Wei Q, Alvarez X, Wang H, Du Y, Zhu H, Jiang H, Zhou J, Lam P, Zhang L, Lackner A, Qin C, Chen Z. Epithelial cells lining salivary gland ducts are early target cells of severe acute respiratory syndrome coronavirus infection in the upper respiratory tracts of rhesus macaques. Journal of virology. 2011; 85(8):4025-30.
Wang H, Zhou M, Brand J, Huang L. Inflammation and taste disorders: mechanisms in taste buds. Annals of the New York Academy of Sciences. 2009; 1170:596-603.
Yukawa M, Naka H, Murata Y, Katayama S, Kohriyama T, Mimori Y, Nakamura S. Folic acid-responsive neurological diseases in Japan. J Nutr Sci Vitaminol (Tokyo). 2001; 47(3):181-7.
Wessels I, Rolles B, Rink L. The Potential Impact of Zinc Supplementation on COVID-19 Pathogenesis. Frontiers in immunology. 2020; 11:1712.
Levy B. D., Clish C. B., Schmidt B, Gronert K, Serhan C. N.. Lipid mediator class switching during acute inflammation: signals in resolution. Nature immunology. 2001; 2(7):612-9.
Serhan C. N. A search for endogenous mechanisms of anti-inflammation uncovers novel chemical mediators: missing links to resolution. Histochemistry and cell biology. 2004; 122(4):305-21.
Bannenberg G. L., Chiang N, Ariel A, Arita M, Tjonahen E, Gotlinger K. H., Hong S, Serhan C. N. Molecular circuits of resolution: formation and actions of resolvins and protectins. Journal of immunology. 2005; 174(7):4345-55.
Serhan C. N., Savill J. Resolution of inflammation: the beginning programs the end. Nature immunology. 2005; 6(12):1191-7.
Serhan C. N., Clish C. B., Brannon J, Colgan S. P., Chiang N, Gronert K. Novel functional sets of lipid-derived mediators with antiinflammatory actions generated from omega-3 fatty acids via cyclooxygenase 2-nonsteroidal antiinflammatory drugs and transcellular processing. The Journal of experimental medicine. 2000; 192(8):1197-204.
Lawrence T, Willoughby D. A., Gilroy D. W.. Anti-inflammatory lipid mediators and insights into the resolution of inflammation. Nature reviews Immunology. 2002;2(10):787-95.
Heasman S. J., Giles K. M., Ward C, Rossi A. G., Haslett C, Dransfield I. Glucocorticoid-mediated regulation of granulocyte apoptosis and macrophage phagocytosis of apoptotic cells: implications for the resolution of inflammation. The Journal of endocrinology. 2003; 178(1):29-36.
Serhan C. N., Brain S. D., Buckley C. D., Gilroy D. W., Haslett C, O'Neill L. A., Perretti M, Rossi A. G., Wallace J. L. Resolution of inflammation: state of the art, definitions and terms. FASEB journal : official publication of the Federation of American Societies for Experimental Biology. 2007; 21(2):325-32.
Kantarci A, Hasturk H, Van Dyke TE. Host-mediated resolution of inflammation in periodontal diseases. Periodontology 2000. 2006; 40:144-63.
Serhan C. N., Hong S, Gronert K, Colgan S. P., Devchand P. R., Mirick G, Moussignac R. L. Resolvins: a family of bioactive products of omega-3 fatty acid transformation circuits initiated by aspirin treatment that counter proinflammation signals. The Journal of experimental medicine. 2002; 196(8):1025-37.
Hong S, Gronert K, Devchand P. R., Moussignac R. L., Serhan C. N.. Novel docosatrienes and 17S-resolvins generated from docosahexaenoic acid in murine brain, human blood, and glial cells. Autacoids in anti-inflammation. The Journal of biological chemistry. 2003; 278(17):14677-87.

Although the foregoing subject matter has been described in some detail by way of illustration and example for purposes of clarity of understanding, it will be understood by those skilled in the art that certain changes and modifications can be practiced within the scope of the appended claims.

1. A method for treating one or more symptoms associated with a severe acute respiratory syndrome coronavirus 2 (SARS-CoV-2) infection and/or Coronavirus disease 2019 (COVID-19), the method comprising administering a therapeutically effective amount of a specialized proresolving mediator (SPM) to a subject in need of treatment thereof to treat the SARS-CoV-2 infection and/or COVID-19.

2. The method of claim 1, wherein the specialized proresolving mediator (SPM) is selected from the group consisting of a lipoxin, a resolvin, a protectin, a maresin, and analogs thereof.

3. The method of claim 2, wherein the specialized proresolving mediator comprises a lipoxin analog.

4. The method of claim 3, wherein the lipoxin analog comprises an analog of one or more of lipoxin A₄ (LXA₄), lipoxin B₄ (LXB₄), 15-epi-lipoxin A₄ (15-epi-LXA₄), and 15-epi-lipoxin B₄ (15-epi-LXB₄).

5. The method of claim 4, wherein the lipoxin analog is selected from the group consisting of: 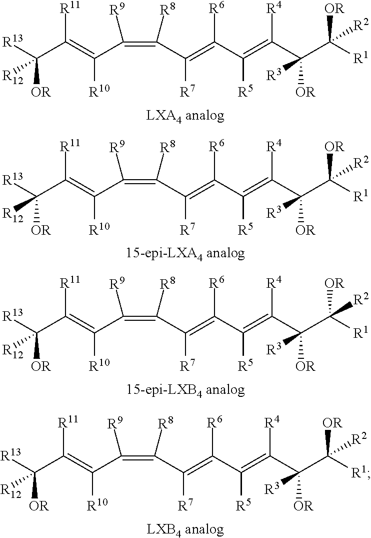
wherein:
R is hydrogen or a straight, branched, cyclic, saturated, or unsaturated alkyl;
R₁, R₂, R₁₂, R₁₃ are each independently selected from hydrogen, straight, branched, cyclic, saturated, or unsaturated alkyl having from 1 to 20 carbon atoms, substituted alkyl having from 1 to 20 carbon atoms, wherein the alkyl is substituted with one or more substituents selected from halo, hydroxy, lower alkoxy, aryloxy, amino, alkylamino, dialkylamino, acylamino, acylamino, hydroxyamino, alkoxyamino, alkylthio, arylthio, carboxy, carboxamido, carboalkoxy, aryl, and heteroaryl, substituted aryl or heteroaryl wherein the aryl or heteroaryl is substituted with one or more substituent selected from alkyl, cycloalkyl, alkoxy, halo, aryl, heteroaryl, carboxyl, and carboxamido, and a group Z-Y, wherein Z is a straight, branched, cyclic, saturated, or unsaturated alkyl having from 1 to 20 carbon atoms; substituted lower alkyl wherein the alkyl is substituted with one or more substituents selected from halo, hydroxy, lower alkoxy, aryloxy, amino, alkylamino, dialkylamino, acylamino, arylamino, hydroxyamino, alkoxyamino, alkylthio, arylthio, carboxy, carboxamido, carboalkoxy, aryl, and heteroaryl, substituted aryl or heteroaryl wherein the aryl or heteroaryl is substituted with one or more substituents selected from alkyl, cycloalkyl, alkoxy, halo, aryl, heteroaryl, carboxyl, and carboxamido, and Y is selected from hydrogen, alkyl, cycloalkyl, carboxyl, carboxamido, aryl, heteroaryl, substituted aryl or heteroaryl wherein the aryl or heteroaryl is substituted with one or more substituents selected from alkyl, cycloalkyl, alkoxy, halo, aryl, heteroaryl, carboxyl, and carboxamido;
R₃ is selected from hydrogen, straight, branched, cyclic, saturated, or unsaturated alkyl having from 1 to 20 carbon atoms, substituted alkyl having from 1 to 20 carbon atoms, wherein the alkyl is substituted with one or more substituents selected from the group consisting of halo, hydroxy, lower alkoxy, aryloxy, amino, alkylamino, dialkylamino, acylamino, arylamino, hydroxyamino, alkoxyamino, alkylthio, arylthio, carboxy, carboxamido, carboalkoxy, aryl, and heteroaryl; substituted aryl or heteroaryl, wherein the aryl or heteroaryl is substituted with one or more substituents selected from the group consisting of alkyl, cycloalkyl, alkoxy, halo, aryl, heteroaryl, carboxyl, and carboxamido; and
R₄, R₅, R₆, R₇, R₈, R₉, R₁₀, and R₁₁ are each independently selected from the group consisting of hydrogen; halo; straight, branched, cyclic, saturated, or unsaturated alkyl having from 1 to 20 carbon atoms; substituted alkyl having from 1 to 20 carbon atoms, wherein the alkyl is substituted with one or more substituents selected from halo, hydroxy, lower alkoxy, aryloxy, amino, alkylamino, dialkylamino, acylamino, arylamino, hydroxyamino, alkoxyamino, alkylthio, arylthio, carboxy, carboxamido, carboalkoxy, aryl, and heteroaryl; substituted aryl or heteroaryl wherein the aryl or heteroaryl are substituted with one or more substituent selected from alkyl, cycloalkyl, alkoxy, halo, aryl, heteroaryl, carboxyl, and carboxamido; or
R, R₁, R₂, R₃, R₄, R₅, R₆, R₇, R₈, R₉, R₁₀, R₁₁, R₁₂, and R₁₃ are connected to form one or more rings containing 3 to 20 carbon atoms, 1 to 6 oxygen atoms or 1 to 6 nitrogen atoms. A pair selected among the R₁, R₂, R₃, R₄, R₅, R₆, R₇, R₈, R₉, R₁₀, R₁₁, R₁₂, and R₁₃ groups can be replaced with a bond that generates a carbon-carbon double or triple bond or a ring.

6. The method of claim 5, wherein the lipoxin analog is an LXA₄ series analog (left column) or a 15-epi-LXA₄ series analog (right column) selected from the group consisting of:

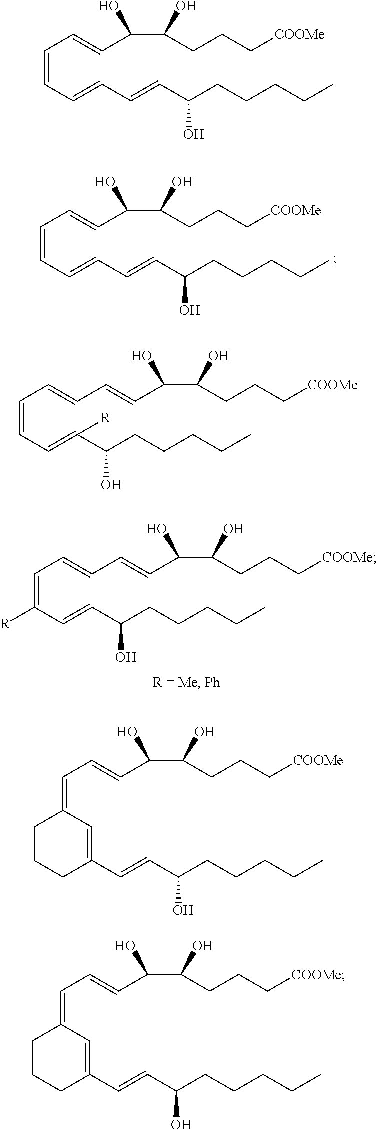

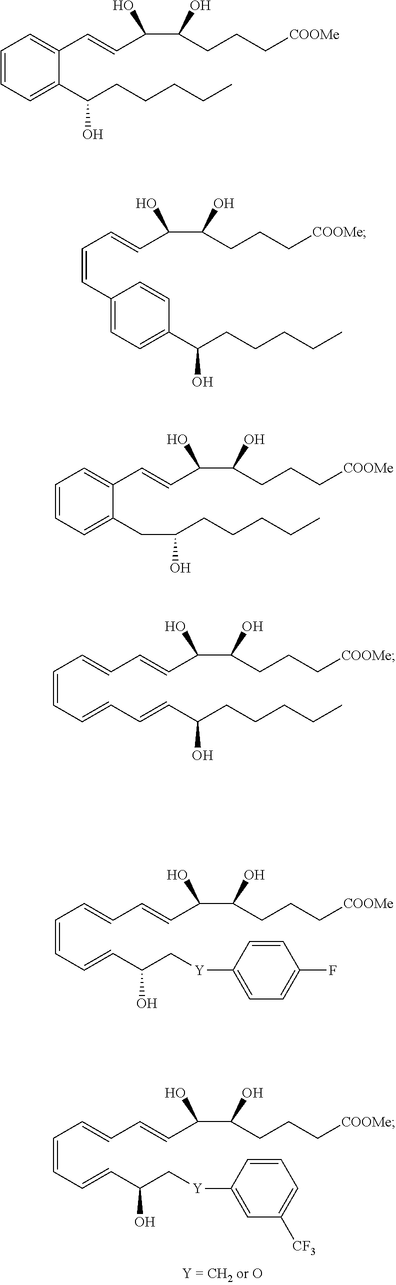

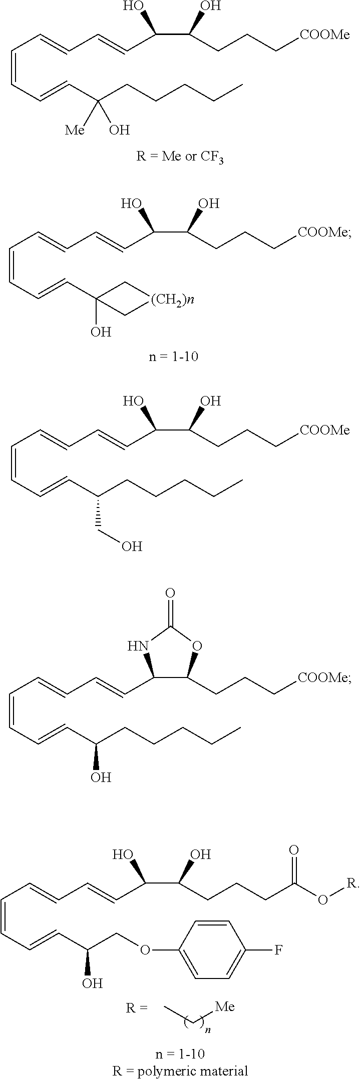

7. The method of claim 5, wherein the lipoxin analog is an LXB₄ series analog (left column) or a 15-epi-LXB₄ series analog (right column) selected from the group consisting of:

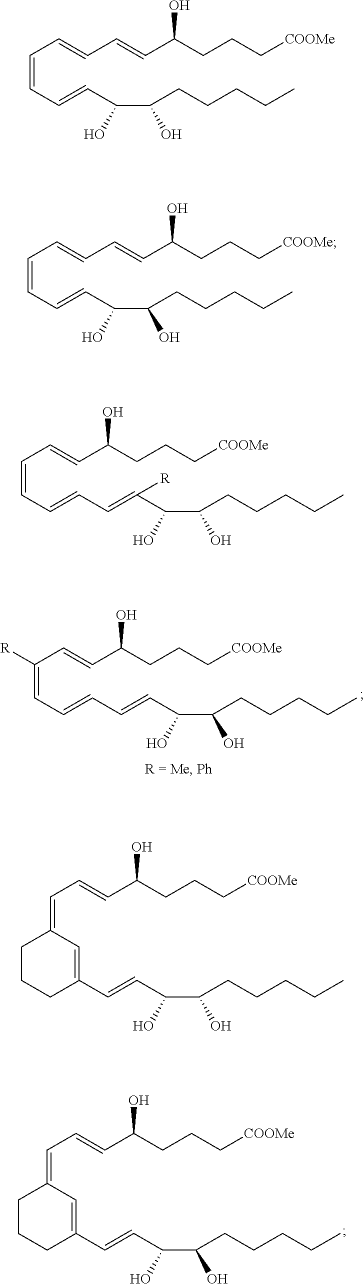

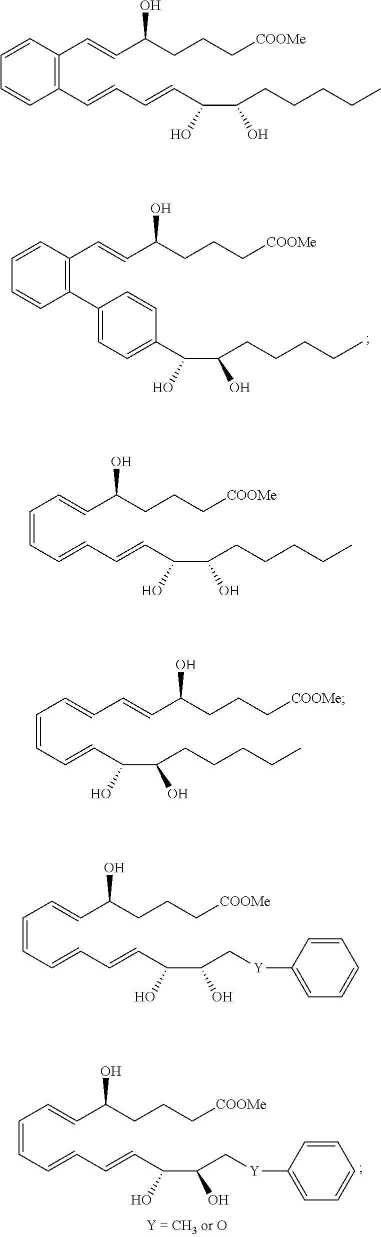

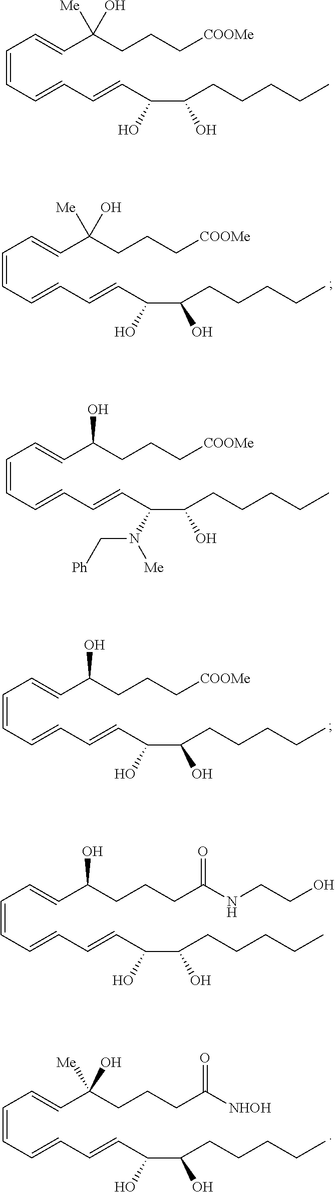

8. The method of claim 5, wherein the lipoxin analog comprises 9,12-benzo-lipoxin A₄ (BLXA₄).

9. The method of claim 5, wherein the lipoxin analog comprises the methyl ester of 9,12-benzo-lipoxin A₄ (BLXA₄-ME).

10. The method of claim 1, wherein the subject is post-COVID-19.

11. The method of claim 1, wherein the one or more symptoms associated with SARS-CoV-2 and/or COVID-19 comprises dysgeusia.

12. The method of claim 11, wherein the dysgeusia includes one or more of hypogeusia, ageusia, and aliageusia.

13. The method of claim 11, wherein the dysgeusia comprises new, early-onset dysgeusia.

14. The method of claim 11, wherein the dysgeusia comprises lingering dysgeusia post-COVID-19.

15. The method of claim 1, wherein the subject experiences a change in taste perception.

16. The method of claim 15, wherein the taste perception of the subject is restored.

17. The method of claim 1, wherein the one or more symptoms associated with severe acute respiratory syndrome coronavirus 2 (SARS-CoV-2) infection or COVID-19 comprises a pulmonary function.

18. The method of claim 17, wherein the pulmonary function comprises dyspnea or fibrosis.

19. The method of claim 1, wherein the specialized proresolving mediator (SPM) is administered orally.

20. The method of claim 19, wherein the specialized proresolving mediator (SPM) is administered as an oral topical rinse.

21. The method of claim 1, comprising administering a dose of the specialized proresolving mediator (SPM) in the range of about 1 to about 20 μg/mL.

22. The method of claim 21, comprising a dose selected from the group consisting of about 1 μg/mL, 5 μg/mL, 10 μg/mL, and 20 μg/mL.
